(19) United States
(12) Patent Application Publication
(10) Pub. No.: US 2025/0389708 A1
(43) Pub. Date: Dec. 25, 2025

(54) TOTAL SOIL CARBON SENSING SYSTEM

(71) Applicant: Soil In Formation, PBC, Saint Louis, MO (US)

(72) Inventors: Shalini Prasad, Allen, TX (US); Vikram Narayanan Dhamu, Dallas, TX (US)

(73) Assignee: Soil In Formation, PBC, Saint Louis, MO (US)

(21) Appl. No.: 18/880,492

(22) PCT Filed: Jun. 30, 2023
(86) PCT No.: PCT/US2023/026662

§ 371 Date: Dec. 31, 2024

Related U.S. Application Data
(60) Provisional application No. 63/357,674, filed on Jul. 1, 2022.

(51) Int. Cl. G01N 33/24; G01N 27/30

(52) U.S. Cl. CPC G01N 33/24; G01N 27/301; G01N 27/307

(57) ABSTRACT

An in-situ, or interfacial, soil sensor is provided, which includes one or more electrodes. Th soil sensor is configured to detect levels of total soil carbon in a sample of soil, the sensor including: a working electrode coated in a composite sensing coating, where the composite sensing coating includes: an active sensing component comprising an organic carbon sensing element functionalized to detect organic carbon moieties and an inorganic carbon sensing element functionalized to detect inorganic carbon moieties, and further includes an encapsulant component.

### CROSS-REFERENCE TO RELATED APPLICATION

This application claims priority to U.S. Provisional Application 63/357,674 filed on Jul. 1, 2022, the content of which is incorporated herein in its entirety.

### BACKGROUND

Soil and water are vital components of the Earth's ecosystem, both playing a major role in maintaining the ecological balance of the planet and sustaining mankind. For instance, the correct use of resources such as water and fertilizers in agriculture application has enormous societal (e.g., health, economic, etc.) importance as well as importance at an environmental level. Governments, corporations, and non-profit groups are working to develop paradigms in which soil and water are protected, regenerated and used more intelligently for the benefit of man and nature. For instance, “regenerative farming” concepts and practices have been proposed and implemented for improving soil health, increasing nutrient levels in crops, improving water use efficiency and reversing climate change through sequestering carbon.

For farmers, the systematic recording of a wide range of information about their fields helps them to implement quality regenerative farming systems, with the aim of improving the information which the farmers rely on for planning and decision-making to increase productivity, land quality, land assets and to reverse losses or inefficiencies in their farms. Optimization of regenerative agriculture is also important at a macro societal level, by protecting crop-producing land, water availability, quality and safety, and reversing negative environmental impacts due to degenerative, inefficient or careless farming practices.

Traditionally, analytical information for soil health has been obtained by means of manually taking soil samples from various accessible zones to represent various soil regions. The samples are then transported to a laboratory to conduct tests and measurements on these samples to better understand the individual nutrient levels in these soil samples and to estimate the overall soil health and water use efficiency in a whole field, farm or in wider regions. Such traditional measurement systems are both inefficient, suboptimal, and expensive, limiting the utility and affordability to the end-user farmers, and thereby limiting the accessibility and momentum needed to implement such “regenerative farming” methods at scale, among other example disadvantages.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 is a simplified block diagram illustrating an example soil sensor system deployed on a plot of land.

FIG. 2 is a simplified block diagram illustrating an example implementation of a soil carbon sensor device.

FIG. 3 is a diagram illustrating example chemical interactions between components of an example composite sensing coating and carbon molecules within an example soil sample.

FIG. 4 is a graph illustrating an example relationship between sensor readings and total soil carbon concentration.

FIG. 5 is a simplified block diagram illustrating an example deployment of a soil sensor system including a soil carbon sensor device.

FIG. 6 is a simplified flow diagram illustrating a technique for using an example soil sensor.

Like reference numbers and designations in the various drawings indicate like elements.

### DETAILED DESCRIPTION

Soil is a fundamental core element of the environment we live in that directly impacts the growth of plants, crops and other vegetation in addition to having a relationship with other parts of the ecosystem including water and air. Better understanding the nature, composition, and health of soil may enable additional solutions to benefit the agricultural initiatives, water quality, and environmental health. Accordingly, it is desirable to reliably collect information related to the physical, chemical and biological components of soil and thereby its vital association with soil health. In some implementations, through the collection of groups of these parameters, the observation of soil and soil-related chemical profiles can be used to create a soil health index with a high degree of reliability in an in-situ manner. For instance, soil chemistry can be derived using in-field probes that have been tuned to generate signals significantly correlated to the physical and biological parameters as well as activity in the soil matrix. Commercially, soil quality determines crop yields and cost of farmland, where it may be essential to have thorough, reliable, dynamic, and in-situ information about soil parameters that are synchronized both in terms of geological location (space) and period (time) due to variations associated with environmental and land use changes. Implementation of dynamic, in-situ soil sensors would significantly benefit the characterization of multiple soil parameters related to soil health from a local as well as global environmental-impact standpoint.

Soil health/quality may be defined by the soil's capacity to function as a sustainable ecosystem that supports plants, animals, and humans alike. Typically, soil health may be characterized according to three main types of soil characteristics: biological, physical, and chemical. Although sometimes used interchangeably, soil quality, from a practical sense, refers to soil chemical and physical properties. For instance, soil health assessment in large part is determined by the nutrient levels in soil. Hence, assessment of soil health parameters in an on-farm setting, facilitates quantification and recording of the soil's inherent physio-chemical and biochemical characteristics. For instance, sufficient levels of soil nutrients may enable sustainable agricultural practices that typically increase the health of the agricultural ecosystem and boost crop yields, pasture growth, etc.

Current attempts to measure soil characteristics involve soil sampling and evaluation techniques that involve intrusive approaches to collect soil samples, through their removal, transportation, and subsequent testing within a laboratory environment that differs from the point of collection. Among these, combustion-based techniques have emerged as one of the most popular techniques for evaluating soil anatomy. Even with recent progress in non-destructive approaches like spectroscopy and tomography-based models, there is a barrier in terms of accuracy (especially at soil depths below 5-10 cm), equipment complexity and availability, as well as high costs and high logistical overhead, among other issues.

There is a wide number of primary, secondary and micronutrients that are required by plants for growth in addition to the dire need for the soil organic matter pool. The rate of nutrient release for uptake by the crops or plants is affected by the availability of the various nutritional sources and other soil parameters. There are a number of electrochemically active and redox substances present in soil that exist in reduced state under ideal conditions (submerged) that contribute to electrochemical activity and in turn have a proportional effect on the soil quality. Some soil properties used to evaluate soil physical properties include bulk density, infiltration parameters, water holding capacity, and soil texture. Soil parameters used for chemical evaluation typically include soil pH, plant available nutrients, soil nitrate, reactive carbon, soil organic matter, and electrical conductivity. Biological properties of soil systems include the diversity and quantity of soil organisms (soil food web), total organic carbon, soil respiration, and soil enzymatic activity. The development of improved sensor systems to successfully and dynamically measure each of these individual parameters can be utilized to develop comprehensive soil health and quality measurements, which may be of substantial benefit to the environment, world food and water supplies, and economic development, among other example benefits.

In accordance with the above, improved sensors may be provided, which leverage soil electrochemistry to correlate soil health in terms of understandable electrochemical signals. Such sensors may be implemented as integrated and miniaturized platforms along with reliable data output. For instance, a sensor device may be implemented as an on-chip in-situ (e.g., deployed on site, within the soil and measuring soil characteristics at the location of the soil sample itself) diagnostic platform for continuously monitoring active parameters inside the dynamic soil ecosystem. Using such sensors, data sets may be collected and processed to identify correlations between electrochemical activity and the presence of active substances that contribute to the soil nutrient cycle. Indeed, such improved in-situ soil sensors may permit soil health to be assessed and monitored at an interfacial level using a probe system. Accordingly, the soil matrix may be characterized using the resulting data in order to provide information in terms of various physio-chemical phenomena occurring at the electrode interface. Subsequently this information can be used to correlate that to useful data which helps to understand soil fertility and bioavailability of nutrients for plants and other vegetation at the field level, among other example insights.

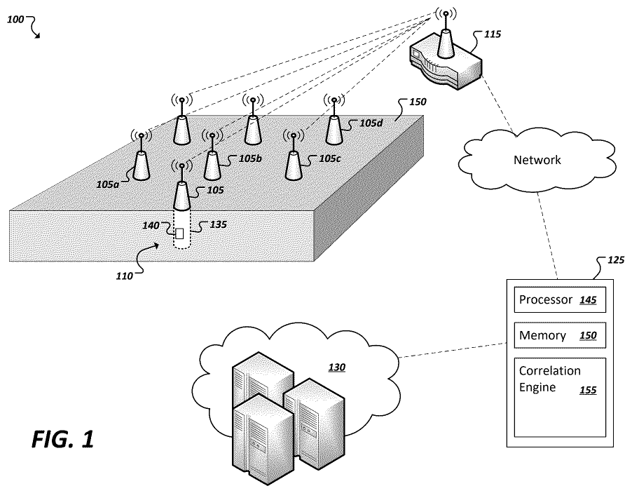

Turning to FIG. 1, a simplified block diagram 100 is shown illustrating an example soil sensing system implemented using one or a collection of computing and/or sensor devices. In one example system, a set of sensors (e.g., 105, 105a-d), such as discussed in the examples below, may be deployed in an agricultural plot 150 to test various volumes, or samples (e.g., 110), of soil at various locations within the plot 150 and/or under various electrochemical modalities to thereby visualize the composite soil chemistry profile of the plot via a point measurement from different characterization perspectives. Such different perspectives may be collected utilizing a collection of sensors (e.g., 105, 105a-d), which include sensors dispersed in various areas of the plot and/or different types of sensors (e.g., measuring the same or varied portions of the plot), as well as collecting measurements from the sensors on a rolling or continuous basis so as to survey the development of the soil's health attributes over time. Thus, improved data and resulting insights may be derived in such in-situ, or in-field, applications due to the sensor devices capturing the dynamic behavior of soil through sensor readings in a range of temporal and spatial settings. Integration of these measurements from a distribution of temporal and spatial points may be used by the system to compute a holistic soil profile for the corresponding region, among other example applications and potential benefits.

In some implementations, supplemental or cooperating computing systems may be provided to communicate with and consume data generated by the collection of sensors (e.g., 105, 105a-d). In one example, a gateway device or other I/O device (e.g., 115) may be utilized to collect signals and other data generated by the sensor devices (e.g., 105, 105a-d) and collect, aggregate, filter, and/or sort the data for consumption by other computing systems and logic. For instance, a computing system (e.g., 125) may be provided with computational logic to determine correlations between the readings of the sensors (e.g., 105, 105a-d) and corresponding soil attributes, which the sensors are configured to measure. For instance, a sensor (e.g., 105) may include one or more electrodes (e.g., 140), which are to contact soil (e.g., 110) and measure electrochemical characteristics of the soil. The electrode(s) 140, in some implementations, may be coated in a specialized coating to enable the electrode 140 to function appropriately within the sensor 105 to enable the sensor to detect total soil carbon within a given soil sample (e.g., 110). For instance, the sensor 105 may generate signals based on these measured electrochemical characteristics. The signals, by themselves, may not directly indicate the level of certain soil health attributes, but through analysis by a correlation engine 155 (e.g., implemented in software and/or hardware of a computing system (e.g., 125)), correlations between certain electrochemical characteristic measurements and corresponding levels of one or more soil health attributes may be determined. While FIG. 1 shows that correlation engine 155 may be executed by a processor 145 and stored in memory 150 of a computing system remote from the sensors (e.g., 105, 105a-d), in some implementations, the hardware and logic of system 125 may be integrated on the sensors themselves to allow this translation between electrochemical readings and various soil health attribute measurements to be determined locally. In some implementations, soil health attribute results determined by a correlation engine 155 may be shared with other computing systems for further storage and/or processing, such as a cloud-based soil-health analysis system (e.g., 130) among other example implementations.

Carbon sequestration refers to different practices that contribute to the absorption and storage of carbon in the soil. While the impact of sequestration of carbon is primarily from organic sources, the effect from soil inorganic carbon (IC) accumulation is not trivial especially in arid and semi-arid regions where the IC share in sequestration is also significant. The potential of efficient carbon sequestration promises improved soil health that increases agricultural productivity and food security values, greater climate resilience (e.g., to potentially combat climate change effects), and reduces fertilizer usage, which brings down the financial burden on farmers and agricultural overhead. To achieve these strategic management practices that will help with carbon sequestration and regenerative farming, it is vital to understand, probe and track soil carbon from these different sources as an aggregate holistic measure. It has been recently understood by scientific communities, environmental agencies, and community users at-large that, while useful, it is not completely sufficient to only track soil organic carbon (SOC) and soil inorganic carbon (SIC), but rather capitalize on also using total soil carbon (TSC) as an objective surrogate measure to keep tabs on carbon sequestration more generally.

In an improved system, sensor devices may be provided with electrodes functionalized to detect soil carbon concentration within a given in-field soil sample in a in situ or point-of-use (PoU) manner. Providing live, in-field sensors represent a significant advancement over state-of-the-art systems, which rely on acquiring samples from the field and removing these samples to a laboratory for testing. Traditional soil testing to monitor and verify the removal or addition of carbon via soil carbon sequestration within an environment is currently difficult and costly. Traditional current soil carbon monitoring is based on extensive soil sampling for analysis in the laboratory or using proximal sensing techniques, which are less accurate. The improved sensing platform introduced herein allows total soil carbon (TSC) to be detected with ease and efficiency and enables live monitoring and data analysis of soil plots in which such sensing platforms are deployed, such as environments illustrated in the example of FIG. 1 and elsewhere.

In some implementations, an improved sensor device may utilize electrodes to capture chemical reactions taking place at the soil sample interface and the resultant electrical energy or signal derived based on these chemical reactions. The corresponding electrical signal is captured and transduced by the sensing device and may be provided to processing logic (e.g., implemented in hardware logic circuitry and/or software/firmware) for interpretation. In some implementations, a sensor device may generate and share the electrical signal generated from the chemical reaction with another device. In other implementations, a sensor device may be further provided with interpretation logic (e.g., based on a trained data analytics model) and may instead or additionally share interpretation results with companion systems (e.g., the interpretation result identifying a TSC level determined by the sensor device) based on an underlying electrical signal generated directly from the chemical reaction captured by the sensor electrodes, among other example implementations.

As noted above, soil carbon may include carbon from both organic and inorganic sources. Organic carbon sources may include the movement and sequestration of atmospheric carbon dioxide (CO2) into plant biomass or other organic matter present in soil and then subsequent conversion of biomass into stable soil organic carbon (SOC) through formation of organo-mineral complexes. Inorganic carbon may include the movement of atmospheric CO2 into soil resources via photosynthesis and root respiration with the subsequent formation of bicarbonate in soil, which exists, for instance, as bicarbonate in solution phase in groundwater sources or mainly precipitates into CaCO3 in soil phase, among other examples. While the above constitute meaningful processes in the global carbon cycle, these two processes correlate to contribute to overall carbon increase in soil, for instance, in accordance with the increase or decrease in microbial activity which is a function of the SOC levels due to enhanced water holding capacity, which may be essential to soil carbonate production and storage. Accordingly, measurement of total soil carbon may be quite useful in assessing present characteristics and trends within a given plot of soil, among other example advantages and considerations.

An improved sensor device may be particularly configured for in-situ sensing with probe integration to survey TSC level within a soil sample in a plot of ground. The readings generated from these sensor devices may be utilized to correlate carbon sequestration and soil quality for agricultural and environmental planning and enhancement, among other example uses. In one example implementation, an improved sensor device may implement a sensor stack based on a modified three-electrode system functionalized towards selective determination of organic and inorganic carbon functional groups in the soil sample matrix. For instance, the sensor may perform interfacial-chemical detection of carbon moieties within the soil sample in which it is inserted or brought into contact using functionalized sensor electrodes with a nanomaterial layer or specialized chemical layer modified to measure aggregate total soil carbon.

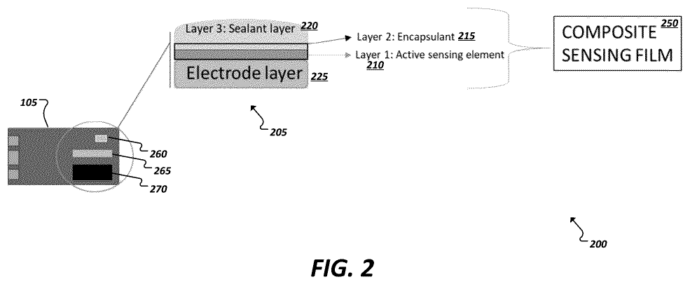

Turning to FIG. 2, a simplified block diagram 200 is shown illustrating an example sensor device 105. In this example, the sensor device 105 includes two or more electrodes. For instance, in this example, a three-electrode configuration is utilized including a counter electrode 270, working electrode 265, and reference electrode 260. FIG. 2 presents a detailed cross-sectional perspective view 205 showing a close-up view of one of the example electrodes of the sensor device 105. More particularly, the cross-sectional view 205 shows the composition of an example composite film coated onto the working electrode 265 to functionalize the working electrode 265 (and sensor device 105) to detect total soil carbon in a soil sample brought into contact with or in proximity to the electrode of the sensor device 105. In one example, the film 250 may include an active sensing element 210, an encapsulant 215, and a sealant 220. One or more of the active sensing element 210, the encapsulant 215, and the sealant 220 may be implemented as a respective layer within the film.

In one example implementation, the functionalized composite sensing film is coated on the working electrode 265 of the sensor device 105 to leverage the interactions between the working electrode 265 and the soil analyte against an uncoated reference electrode 260. In another example, the working electrode 265 is functionalized by applying the active sensing element 210 and encapsulant 215 to the working electrode 265 only, with the sealant layer 220 applied to coat each of the working electrode 265, reference electrode 260, and counter electrode 270 and act as a support electrolyte for electrochemical transduction, among other example implementations using a composite sensing film configured for detection soil carbon.

An example composite sensing film may be functionalized to include materials, or sensing elements, corresponding to the detection of organic carbon and inorganic carbon. More particularly, the active sensor element may be selected based on particular interactions between the modified electrode and organic carbon and inorganic carbon content in the soil that can be determined by applying an experimentally determined input bias and process, corresponding to the OC and IC fraction specifically. In some implementations, the components or coatings utilized to form the composite sensing film 250 selectively interact and functionalize the detection of the organic fraction of soil carbon that is triggered with an input signal that drives a chemical reaction by perturbing the electrode-soil double layer interface and captures charge movement and modulation as a function of organic carbon in the soil system. These corresponding electrochemical kinetics-driven signal changes are used to quantify and monitor changes to labile and recalcitrant organic carbon (OC). This combination of film components may also be leveraged to detect different carbonate and bicarbonate ionic entities in soil, which may be accounted for by operating the sensor system with a particular input bias (e.g., as determined experimentally) in order to interact and capture the complete profile of inorganically sourced carbon-based minerals (IC) in the soil sample.

In one example implementation, a first active sensing element layer may be deposited or otherwise applied to the exterior surface of an electrode layer 225. The active sensing element may be composed of nanomaterial structures, ion correlated liquids, covalent capture frameworks integrated with organic compounds, such as compounds based on pyridines or calixarenes (for ion capture), which promote binding/interaction that is captured using electrochemical modes to track soil carbon analogues, among other examples. The active sensing element may serve as the main sensing element of the system. For an example encapsulant layer, in one example implementation, a diatomaceous earth, active carbon powder may be utilized to hold the active element of the sensing structure while promoting nutrient interaction and retention properties in soil that aid in capturing soil mineral groups. More generally, the encapsulant layer or material 215 may include an amorphous carbon-based or derived material that can be blended with the active sensing element material (210) into a cement- or putty-like substance and coated onto the sensor electrode. This putty like structure acts as a pre-cursor to serve as the main interaction site/layer between the electrode and the soil analyte medium. In some implementations, materials of the active element layer 210 and encapsulated layer 215 may be homogenously mixed into a composite pre-cursor (e.g., by stirrer and pipette-mixing). Such a composite pre-cursor may serve as the functional component of the film and represent where the sensing and interaction sites are present to leverage the physico-chemical reaction between the sensor and soil analyte to account for soil carbon within a given sample. In one example, the sealant layer 220 may be composed of polymeric entities (e.g., polyvinyl carbonite (PVC), polymethyl methacrylate (PMMA), polyaniline, poly (3,4-ethylenedioxythiophene) polystyrene sulfonate (PEDOT:PSS), etc.). The sealant layer material may be added into the composite coating precursor to act as a film sealant that holds the composite against the electrode layer in a functionalized manner and thereby act as a support electrolyte layer in this modified electrochemical sensor structure. In one example implementation, the composite material of the composite sensing film (e.g., collectively layers 1, 2 and 3) may be mixed to form a gel-like physical structure by pipette-action, then sonicated (e.g., for 15 minutes) and vortexed (e.g., for 5 minutes). This gel-like composite ink may then be coated onto the sensor by drop-casting or spin-coating to form a functionalized layer on working electrode of the 3-layer electrode system.

While some of the examples discuss the components of the composite sensing film 250 as “layers” it should be appreciated that such layers represent the composite sensing film's 250 component ingredients and may not be physically disposed on electrodes as layers. For instance, in some implementations, the active sensing element 210 and encapsulant 215 may be mixed together into a pre-cursor composite (e.g., a putty-like substance) and coated as a mixture onto sensor electrodes to create an interaction layer. This interaction layer may be enhanced with a secondary membrane network with the addition of the sealant layer 220 added on top of the pre-cursor composite. This forms a layer-by-layer stack with 210 and 215 as the base stack and 220 sealant membrane as the second level stack. In another example implantation, the pre-cursor composite (formed from a blend of the active sensing element 210 and encapsulant 215) may be mixed with the sealant material 220 to form a composite mixture concoction that is coated onto the sensor forming a single layer network comprising of differently sub-layered elements (e.g., 210, 215 and 220), among other example implementations of the composite sensing film 250.

As another example of composite coating, which may be deposited on the working electrode of an example sensor device to functionalize the sensor device for the detection or measurement of TSC concentrations, a coating composition may utilize a composite base system for assessing organic and inorganic carbon groups present in a soil sample brought into contact with the sensor device's electrode. In one example, mixture of inorganic carbon sensing elements and organic carbon sensing elements may be brought together (e.g., mixed) and dispensed within a buffer suspension. In one example, the organic carbon sensing element may be implemented using a room temperature ionic liquid (RTIL) formulated to interact with organic carbon moieties (e.g., active organic matter (AOM) and humic substances (HS)). For the inorganic carbon sensing element, in one example implementation, a calixarene-based compound may be applied for interaction with carbonate (CO3) moieties. The organic carbon sensing compound and inorganic carbon sensing compound may be provided in a solution at different concentrations and ratios. For instance, in one example implementation, the composite coating layer may be based on a 1:4 ratio, with one part (e.g., by weight) of the organic carbon sensing element to four parts of the inorganic carbon sensing element. These quantities of organic and inorganic carbon sensing elements may be dispensed in a suspension (e.g., chloroform) to form a composite base.

In one example, the sensing element composite base may be combined with a solution providing a polymer membrane network, on which the active sensing layer may be disposed, allowing soil to penetrate the sensing layer and interact with the organic and inorganic carbon sensing elements in the composition. In one example, a chitosan solution may be utilized to implement the polymer membrane network. In one example, 5 μL of high molecular weight chitosan solution may be added to 20 uL of the organic-inorganic-composite solution to form (e.g., 25 uL) of a composite coating for sensing TSC. In some implementations, the composite coating may form an “ink” composition, capable of being screen-printed, drop-casted, or otherwise deposited on a working electrode of a TSC sensor device and functionalize the working electrode for TSC detection. Table 1 below illustrates characteristics of an example coating. For instance, an organic carbon sensing element may be implemented as an RTIL, such as [EMIM TF2N], among other examples:

TABLE 1
Example Down-Selected Elements for Coating
and Measurements for a TSC Sensor

| | | Electro- | |
|---|---|---|---|
| | | chemical | |
| RTIL | Conductivity | Window | Physical Property |
| EMIM | 6.63 mS/cm | 4.7 V | Aromatic, weakly coordination |
| TF2N | | | anion, hydrophobic, viscosity 40 cP |

The electrochemical reactions may be observed using an example sensor device, such as discussed in the examples above. For instance, a two-pronged conjunctive approach may be utilized to concurrently detect electrochemical attributes associated with the presence of both organic carbon and inorganic carbon in a sample. From these two approaches, a complete picture of the total carbon composition of a sample may be determined. For instance, in a first, electrochemical impedance spectroscopy-based approach, impedimetric double layer analysis and modelling may be utilized to study the interactions at the sensing film-soil carbon (OC and IC) interface and correlate modulations towards total soil carbon levels (e.g., using electrochemical impedance spectroscopy (EIS) analysis). For instance, the interface formed between the soil system (analyte) and the functionalized sensor may be probed using an impedance-based detection technique, where the presence of varied levels of soil carbon correlate to corresponding changes/modulation in impedance signals generated at the sensor device. For instance, in one example, an EIS technique may be used by applying a small AC input bias, and scanning the impedance spectra across frequencies, to map different chemical reactions and interactions that occur at the sensor-soil interface. For instance, the voltage may be applied and swept between the working and reference electrodes of the sensor device, with the resulting current formed at the working electrode measured as an output. In such example, at a particular frequency (e.g., which may be experimentally obtained by scanning across a larger frequency range), the chemical interactions caused between the functionalized sensor layer and the soil analyte causes an impedance modulation. In one example, a 10 mV AC voltage input is scanned from 100 MHx to 1 Hz. Accordingly, measurements at this selected, particular frequency represent the area of interest and the impedance signal at this frequency is extracted as an output corresponding to varied levels of soil carbon which cause modulations to the output impedance. This particular frequency of interest also corresponds to whether the physico-chemical reaction measured between the sensor and soil is causing more resistive modulations, which calls for frequency of interest in the higher, range or whether the interactions cause capacitive/diffusion-based changes, which show up in the lower end of the frequency range. Accordingly, it is possible experimentally to obtain where the frequency signature is for output impedance extraction as a function of soil carbon changes.

A second, cyclic voltammetry approach may be utilized to apply a pulsed direct current (DC) signal (e.g., with AC auxiliary input) at the sensor device (e.g., between the working and reference electrodes of the sensor device) to perturb the sensing film and capture the polarized interaction layer formed between the electrode layer and the soil carbon groups that correspond to the signal amplitude to TSC concentration (e.g., using a differential pulse voltammetry (DPV) and linear sweep voltammetry) approach. For instance, the interface formed between the soil analyte and the functionalized sensor may be excited by applying a pulsed voltage signal as an input. For instance, a −1V to +1V sweep may be applied at a 100 mV/sec rate, among other examples. Next, the observed peak currents are recorded, which form due to the induced activity (e.g., due to the excitement voltage) of the soil carbon entities at the same interface. Based on the concentration of soil carbon molecules present in the soil sample, the level of activity will be modulated and thereby the peak current levels will vary. This peak current may be extracted and correlated with soil carbon levels to determine the input-output translation between the signals generated by the sensor in response and the corresponding TSC level in the soil sample. In some implementations, a combination of both the cyclic voltammetry- and the electrochemical impedance-based approaches may be utilized to derive a multi-variate relationship to TSC concentration within a sample, among other examples. Accordingly, the impedance and/or current generated by a TSC-functionalized sensor device may be read and utilized to derive a corresponding TSC level. In other cases, a single one of the impedance or the current generated by the sensor device may be relied upon and mapped to a corresponding TSC level, among other examples.

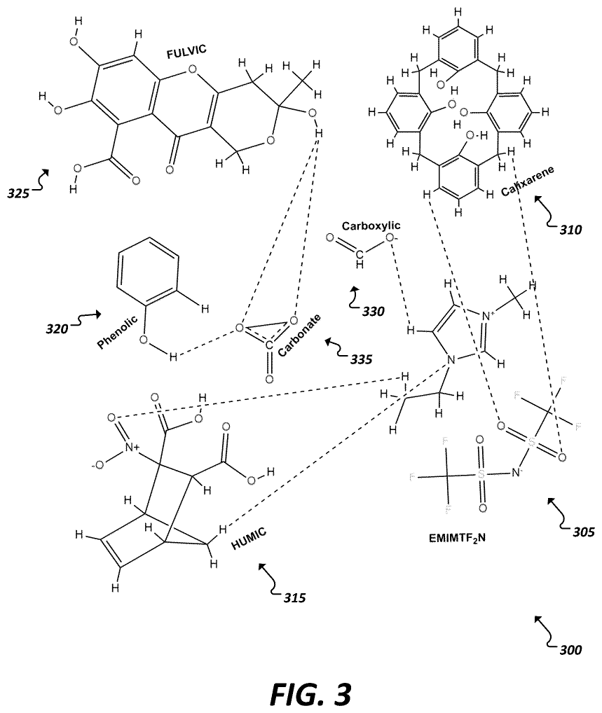

FIG. 3 is a diagram 300 illustrating example interactions between an RTIL-based organic carbon sensing element 305 (e.g., [EMIM TF2N]) and a calixarene-based inorganic carbon sensing element 310 with a variety of different carbon chemical moieties, which may be present within a soil sample brought into contact with an electrode functionalized for detecting TSC levels. For instance, in this example, a complex formulation of organic and inorganic soil components (e.g., 315, 320, 325, 330, 335, etc.) may interact with the sensing elements 305, 310. For instance, the soil carbon components may interact with the cations of the RTIL-based organic carbon sensing element 305 and the anions of the RTIL-based organic carbon sensing element 305 may interact with the calixarene-based inorganic carbon sensing element 310, which further interface with the fulvic acid-base components (e.g., 330) and carbonate entities (e.g., 335) to holistically form a carbon complex that may represent the total soil carbon within a sample. The carbon complex, in this example, includes eight hydrogen bonds. The corresponding thermodynamic energy may correspond with the spontaneity and ease with which TSC compounds interact with the functionalized TSC sensor coating or layer. For instance, large numbers of hydrogen bonding interactions and higher spontaneity are indicated by high negative free energy for interactions and bond formation with TSC compounds in soil.

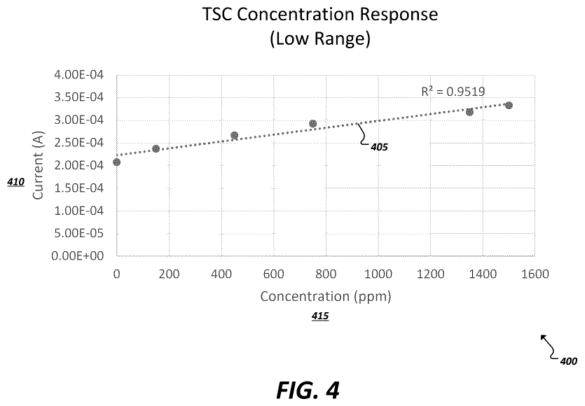

Turning to FIG. 4, a graph 400 is shown illustrating a relationship between TSC concentration response and a current value measured, in one example, between electrodes on a sensor device functionalized to have its working electrode interact with both inorganic and organic carbon moieties. In this example, the composite active layer deposited on the working electrode enables chemical interactions that result in a correlation 405 (e.g., linear correlation) between the current (e.g., 410) generated at the sensor device and the concentration of TSC (e.g., 415) within a sample. A soil sensing system may utilize such a correlation to derive a TSC level from signals generated by TSC sensor devices deployed in various soil samples within a plot.

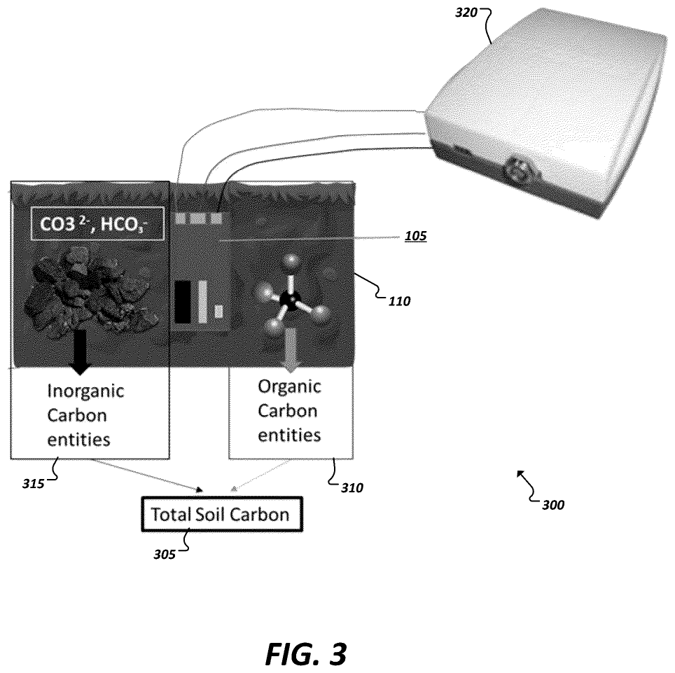

Turning to FIG. 5, a simplified block diagram 500 is shown illustrating an example soil sensor system. For instance, an enhanced soil sensor system configured to detect TSC levels, such as discussed in the examples above, may include a sensor device 105 with one or more sensor electrodes functionalized as discussed in the examples above, which are inserted or deployed into the soil 110 to detect soil carbon entities in the soil sample, including the aggregate total soil carbon level 505 comprised of both organic carbon levels 310 and inorganic carbon levels 515, for instance, using a composite coating applied to the working electrode (e.g., only the working electrode) of the sensor device 105. In some implementations, the soil sensor devices may be hardwired or connect wirelessly (e.g., via an integrated wireless communication module) to supporting hardware (e.g., 520) capable of recording or performing analytics on the data generated by and received from the sensor devices. In some implementations, such systems may be locally deployed. In other implementations, such systems may include cloud-based computing systems (e.g., which the sensor devices may communicate with via a local gateway devices). In still other implementations, data storage and analytics/interpretation logic may be included on the sensor devices, among other example implementations. As one example, sensor devices may connect to a potentiostat system (e.g., a portable or battery-powered system) capable of performing calculations on measurements obtained from the sensor devices (e.g., discussed in the first and second approaches above), as well as recording and analyzing output data.

While the examples above illustrate example soil sensor implementations, it should be appreciated that these are presented as illustrative examples only and that a variety of other, additional interfacial soil sensors may be implemented based on and applying the principles described herein, including sensors with varying form factors and substrates, sensors applying different active, encapsulant, and/or sealant layers or coatings, and sensors capable of being used to measure other attributes of soil health (e.g., other soil organic matter compounds). Moreover, multiple sensor designs may be applied and integrated within a single sensor device to enable the device to concurrently measure multiple different soil health attributes for a corresponding soil sample matrix and multi-variant analysis of the subject soil. Indeed, an array of soil health attributes may be advantageously measured using soil sensor systems such as described herein to develop measurements of the overall health of a plot of ground (e.g., farmland, ranch land, orchard plots, vineyards, and the like).

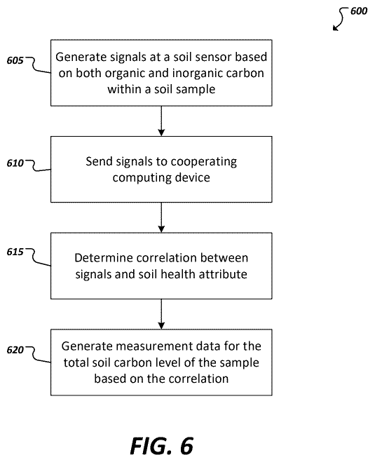

FIG. 6 is a simplified flow diagram 600 illustrating an example technique involving the use of an example in-situ soil sensor. The sensor may be deployed in a particular soil sample (either isolated in a container or representing a portion of a large plot of ground or soil). Electrodes of the soil sensor may be in prolonged and direct contact with the soil and may be configured to react to, measure, or detect chemical properties of the soil based on electrochemical reactions measured at the electrodes of the sensor. The sensor, through the electrodes, may generate signals based on a composite sensing coating applied to the electrodes of the sensor. The coating may include an active sensing element composed of a composite of inorganic sensing elements and organic carbon sensing elements. Further, the coating may further include encapsulant and sealant elements. A sensor with electrodes (e.g., its working electrode) bearing this coating may be enabled to generate signals 605 corresponding to a carbon complex concentration (of both the inorganic and organic carbon levels) of the soil. The signals may be sent 610 to a cooperating computing device, which include computer processing hardware and logic to determine 615 correlations between the generated signals and the organic and inorganic carbon levels of the soil sample. In some implementations, the cooperating computing device may be different from and remote from the sensor device. In other implementations, the computing device and its hardware may be integrated with the sensor device and deployed together in the field. Measurement data may be generated 620 based on the determined correlation to indicate a measure of the total soil carbon level at the soil sample. This information may be further used, stored, shared, or tracked to assess, on a continuing basis, the total soil carbon levels of and carbon sequestration in this portion of the soil, and through the deployment of multiple such sensors in multiple nearby soil samples, the overall soil carbon attributes of a plot of land and its soil, among other example applications and benefits.

Note that in this document, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments. Furthermore, the words “optimize,” “optimization,” and related terms are terms of art that refer to improvements in speed and/or efficiency of a specified outcome and do not purport to indicate that a process for achieving the specified outcome has achieved, or is capable of achieving, an “optimal” or perfectly speedy/perfectly efficient state.

In general, computing systems, which interface with a biosensor via a wired or wireless communication channel, can include electronic computing devices operable to receive, transmit, process, store, or manage data and information associated with the biosensor and other subsystems of the computing system. As used in this document, each of the terms “computer,” “processor,” “processor device,” “microcontroller,” or “processing device” is intended to encompass any suitable data processing apparatus. For example, while the microcontroller may be implemented, in some examples, as a single device within the computing system, in other implementations the processing functionality of the system may be implemented using a plurality of computing devices and processors, such as a fog computing system, server pools, a cloud computing system, or other distributed computing system including multiple computers. Further, any, all, or some of the computing devices may be adapted to execute any operating system, including Linux, UNIX, Microsoft Windows, Apple OS, Apple IOS, Google Android, Windows Server, etc., as well as virtual machines adapted to virtualize execution of a particular operating system, including customized and proprietary operating systems.

In some implementations, all or a portion of a computing platform may function as a wearable device, standalone biosensor device, or other sensor device. A sensor device may connect to and communicate with other computing devices through wired or wireless network connections. For instance, wireless network connections may utilize wireless local area networks (WLAN), such as those standardized under IEEE 802.11 family of standards, home-area networks such as those standardized under the Zigbee Alliance, personal-area networks such as those standardized by the Bluetooth Special Interest Group, cellular data networks, such as those standardized by the Third-Generation Partnership Project (3GPP), and other types of networks, having wireless, or wired, connectivity. For example, an endpoint device may also achieve connectivity to a secure domain through a bus interface, such as a universal serial bus (USB)-type connection, a High-Definition Multimedia Interface (HDMI), or the like.

It is also important to note that the operations and steps described with reference to the preceding FIGURES illustrate only some of the possible scenarios that may be executed by, or within, the system. Some of these operations may be deleted or removed where appropriate, or these steps may be modified or changed considerably without departing from the scope of the discussed concepts. In addition, the timing of these operations may be altered considerably and still achieve the results taught in this disclosure. The preceding operational flows have been offered for purposes of example and discussion. Substantial flexibility is provided by the system in that any suitable arrangements, chronologies, configurations, and timing mechanisms may be provided without departing from the teachings of the discussed concepts.
The following examples pertain to embodiments in accordance with this Specification. Example 1 is an apparatus including: a sensor to detect levels of total soil carbon in a sample of soil, the sensor including: a working electrode coated in a composite sensing film, where the composite sensing film includes: an active sensing component functionalized to detect both organic carbon moieties and inorganic carbon moieties; an encapsulant component; and a sealant component; and another electrode.
Example 2 includes the subject matter of example 1, where the encapsulant component includes a material to promote capture of mineral groups from the soil sample.
Example 3 includes the subject matter of any one of examples 1-2, where the sealant component acts as a support electrolyte for electrochemical transduction.
Example 4 includes the subject matter of any one of examples 1-2, where the other electrode includes a reference electrode.
Example 5 includes the subject matter of example 4, where the sensor further includes a counter electrode.
Example 6 includes the subject matter of example 5, where the composite sensing film is layered over the working electrode and the reference electrode and a sealant layer including the sealant component is coated on the counter electrode.
Example 7 includes the subject matter of example 6, where a composite layer including a mixture of the active sensing component and the encapsulant component is deposited on the working electrode and the reference electrode, and the sealant layer is also deposited over the composite layer to form the composite sensing film.
Example 8 includes the subject matter of example 5, where the composite sensing film is layered over each of the working electrode, the reference electrode, and the counter electrode.
Example 9 includes the subject matter of any one of examples 1-8, further including circuitry to: apply a voltage at the sensor; and detect impedance at the sensor based on presence of organic or inorganic soil carbon in the soil sample.
Example 10 includes the subject matter of example 9, where the voltage includes a pulsed voltage signal applied across the working electrode and reference electrode.
Example 11 includes the subject matter of example 10, where the pulsed voltage signal is applied according to a particular frequency associated with detection of varied levels of soil carbon.
Example 12 includes the subject matter of example 9, further including a communication module to send a signal to another computing device to communicate the detected impedance.
Example 13 includes the subject matter of any one of examples 1-12, where the sensor includes an in-situ soil sensor.
Example 14 is a method including: applying a voltage across a working electrode and reference electrode of an in-situ soil sensor deployed in a soil sample, where the working electrode is coated with a composite sensing film including an active sensing component, an encapsulant component, and a sealant component; and generating impedance signals at the in-situ soil sensor, where the impedance signals are generated based on concentration of inorganic soil carbon and organic soil carbon in the soil sample, where the active sensing component is configured to detect both organic carbon moieties and inorganic carbon moieties.
Example 15 includes the subject matter of example 14, further including determining, from the impedance signals, a concentration of total soil carbon within the soil sample.
Example 16 includes the subject matter of example 15, further including transmitting a signal to another computing device to identify the impedance signals to the other computing device, where the other computing device determines the concentration of total soil carbon within the soil sample.
Example 17 is a system including means to perform the method of any one of examples 14-16.
Example 18 is a system including: a sensor device including: a plurality of electrodes, where the plurality of electrodes includes a working electrode coated in a composite sensing film, where the composite sensing film includes an active sensing component functionalized to detect both organic carbon moieties and inorganic carbon moieties, an encapsulant component, and a sealant component; and circuitry to generate an impedance based on concentration of total soil carbon in a soil sample when in contact with the plurality of electrodes, where the total soil carbon includes organic soil carbon and inorganic soil carbon. The system further includes an analysis system including: a processor; and analytics logic executable by the processor to determine, from the impedance, a value of the concentration of total soil carbon in the soil sample.
Example 19 includes the subject matter of example 18, further including a plurality of sensor devices deployed in a plurality of soil samples within an environment.
Example 20 includes the subject matter of example 18, where the sensor device includes an in situ soil sensor.
Example 21 includes the subject matter of example 18, where the sensor device includes the apparatus of any one of examples 1-13.
Example 42 is an apparatus including: a sensor to detect levels of total soil carbon in a sample of soil, the sensor including: a working electrode coated in a composite sensing coating, where the composite sensing coating includes: an active sensing component including an organic carbon sensing element functionalized to detect organic carbon moieties and an inorganic carbon sensing element functionalized to detect inorganic carbon moieties; and an encapsulant component; and another electrode.
Example 43 includes the subject matter of example 42, where the organic carbon sensing element includes a room temperature ionic liquid (RTIL).
Example 44 includes the subject matter of example 43, where the RTIL includes [EMIMTF2N].
Example 45 includes the subject matter of any one of examples 42-44, where the inorganic carbon sensing element includes calixarene.
Example 46 includes the subject matter of any one of examples 42-45, where the composite sensing coating further includes a sealant component.
Example 47 includes the subject matter of any one of examples 42-46, where the encapsulant component includes a material to promote capture of mineral groups from the soil sample.
Example 48 includes the subject matter of example 47, where the encapsulant component includes a polymer membrane network.
Example 49 includes the subject matter of any one of examples 42-48, where composite sensing coating further includes a sealant components.
Example 50 includes the subject matter of example 49, where the sealant component acts as a support electrolyte for electrochemical transduction.
Example 51 includes the subject matter of any one of examples 42-50, where the other electrode includes a reference electrode.
Example 52 includes the subject matter of example 51, where the sensor further includes a counter electrode.
Example 53 includes the subject matter of any one of examples 51-52, where the composite sensing coating is only layered over the working electrode.
Example 54 includes the subject matter of example 53, where a composite layer including a mixture of the active sensing component and the encapsulant component is deposited on the working electrode and a sealant layer is deposited over the composite layer to form the composite sensing coating.
Example 55 includes the subject matter of any one of examples 42-54, further including circuitry to: apply an alternating current (AC) voltage; and detect impedance at the sensor based on presence of a carbon complex including organic carbon and inorganic carbon in the soil sample.
Example 56 includes the subject matter of example 55, where the voltage is applied across the working electrode and the other electrode.
Example 57 includes the subject matter of example 56, where the voltage is applied according to a particular frequency associated with detection of varied levels of soil carbon.
Example 58 includes the subject matter of any one of examples 56-57, further including circuitry to: apply a DC voltage; and detect current at the sensor based on presence of a carbon complex including organic carbon and inorganic carbon in the soil sample.
Example 59 includes the subject matter of any one of examples 56-58, further including a communication module to send a signal to another computing device to communicate the detected impedance or current.
Example 60 includes the subject matter of any one of examples 42-59, where the sensor includes an in-situ soil sensor.
Example 61 is a method including: applying a voltage across a working electrode and reference electrode of an in-situ soil sensor deployed in a soil sample, where the working electrode is coated with a composite sensing coating including an active sensing component, an encapsulant component, and a sealant component; and generating impedance signals at the in-situ soil sensor, where the impedance signals are generated based on concentration of inorganic soil carbon and organic soil carbon in the soil sample, where the active sensing component is configured to detect a concentration of a carbon complex in the soil sample, and the carbon complex includes both organic carbon moieties and inorganic carbon moieties.
Example 62 includes the subject matter of example 61, further including determining, from the impedance signals, a concentration of total soil carbon within the soil sample.
Example 63 includes the subject matter of example 62, further including transmitting a signal to another computing device to identify the impedance signals to the other computing device, where the other computing device determines the concentration of total soil carbon within the soil sample.
Example 64 includes the subject matter of any one of examples 61-63, where the active sensing component includes an organic carbon sensing element functionalized to detect organic carbon moieties and an inorganic carbon sensing element functionalized to detect inorganic carbon moieties.
Example 65 includes the subject matter of example 64, where the organic carbon sensing element includes a room temperature ionic liquid (RTIL).
Example 66 includes the subject matter of example 65, where the RTIL includes [EMIMTF2N].
Example 67 includes the subject matter of any one of examples 64-66, where the inorganic carbon sensing element includes calixarene.
Example 68 includes the subject matter of any one of examples 64-67, where the active sensing component includes a polymer membrane network.
Example 69 is a system including means to perform the method of any one of examples 61-68.
Example 70 is a composite coating to interact with a carbon complex including inorganic carbon and organic carbon, where the composite coating includes: an organic carbon sensing element functionalized to detect organic carbon moieties; an inorganic carbon sensing element functionalized to detect inorganic carbon moieties; and a polymer membrane network.
Example 71 includes the subject matter of example 70, where the organic carbon sensing element includes a room temperature ionic liquid (RTIL).
Example 72 includes the subject matter of example 71, where the RTIL includes [EMIMTF2N].
Example 73 includes the subject matter of any one of examples 70-72, where the inorganic carbon sensing element includes calixarene.
Example 74 includes the subject matter of any one of examples 70-73, including a mixture of the organic carbon sensing element, the inorganic carbon sensing element, and the polymer membrane network.
Example 75 includes the subject matter of any one of examples 70-74, where the composite coating is adapted for screen printing or drop casting onto a surface of a sensor electrode.
Example 76 includes the subject matter of any one of examples 70-75, further including a sealant layer.
Example 77 is a system including: a sensor device including: a plurality of electrodes, where the plurality of electrodes includes a working electrode coated in a composite sensing coating, where the composite sensing coating includes an active sensing component functionalized to detect both organic carbon moieties and inorganic carbon moieties and an encapsulant component, and a sealant component; and circuitry to generate a signal to identify an impedance or a current based on concentration of total soil carbon in a soil sample when in contact with the plurality of electrodes, where the total soil carbon includes organic soil carbon and inorganic soil carbon; and an analysis system including: a processor; and analytics logic executable by the processor to determine, from the impedance, a value of the concentration of total soil carbon in the soil sample.
Example 78 includes the subject matter of example 77, further including a plurality of sensor devices deployed in a plurality of soil samples within an environment.
Example 79 includes the subject matter of any one of examples 77-78, where the sensor device includes an in situ soil sensor.
Example 80 includes the subject matter of any one of examples 77-79, where the analysis system is to collect a series of readings over time and determine changes in the concentration of the total soil carbon from the series of readings.
Example 81 includes the subject matter of any one of examples 77-80, where the active sensing component includes a composite of an organic carbon sensing element and an inorganic carbon sensing element, and the organic carbon sensing element includes a room temperature ionic liquid (RTIL).
Example 82 includes the subject matter of example 81, where the RTIL includes [EMIMTF2N].
Example 83 includes the subject matter of any one of examples 81-82, where the inorganic carbon sensing element includes calixarene.
Example 84 includes the subject matter of any one of examples 77-83, where the sealant component includes a polymer membrane network.
Example 85 includes the subject matter of example 77, where the sensor device includes the apparatus of any one of examples 42-60.

Although the present disclosure has been described in detail with reference to particular arrangements and configurations, these example configurations and arrangements may be changed significantly without departing from the scope of the present disclosure. Numerous other changes, substitutions, variations, alterations, and modifications may be ascertained to one skilled in the art and it is intended that the present disclosure encompass all such changes, substitutions, variations, alterations, and modifications as falling within the scope of the appended claims.

1-44. (canceled)

45. An apparatus comprising:
a sensor to detect levels of total soil carbon in a sample of soil, the sensor comprising:
a working electrode coated in a composite sensing coating, wherein the composite sensing coating comprises:
an active sensing component comprising an organic carbon sensing element functionalized to detect organic carbon moieties and an inorganic carbon sensing element functionalized to detect inorganic carbon moieties; and
an encapsulant component comprising a polymer membrane network; and
another electrode, wherein the sensor comprises an in-situ soil sensor.

46. The apparatus of claim 45, wherein the organic carbon sensing element comprises a room temperature ionic liquid (RTIL).

47. The apparatus of claim 46, wherein the RTIL comprises [EMIMTF2N].

48. The apparatus of claim 45, wherein the inorganic carbon sensing element comprises calixarene.

49. The apparatus of claim 45, wherein composite sensing coating further comprises a sealant component, and the sealant component acts as a support electrolyte for electrochemical transduction.

50. The apparatus of claim 45, wherein the other electrode comprises a reference electrode and a counter electrode, and the composite sensing coating is only layered over the working electrode.

51. The apparatus of claim 45, further comprising circuitry to:
apply an alternating current (AC) voltage; and
detect impedance at the sensor based on presence of a carbon complex comprising organic carbon and inorganic carbon in the soil sample, wherein the voltage is applied across the working electrode and the other electrode.

52. The apparatus of claim 51, wherein the voltage is applied according to a particular frequency associated with detection of varied levels of soil carbon.

53. A method comprising:
applying a voltage across a working electrode and reference electrode of an in-situ soil sensor deployed in a soil sample, wherein the working electrode is coated with a composite sensing coating comprising an active sensing component, an encapsulant component, and a sealant component, wherein the active sensing component comprises an organic carbon sensing element functionalized to detect organic carbon moieties and an inorganic carbon sensing element functionalized to detect inorganic carbon moieties; and
generating impedance signals at the in-situ soil sensor, wherein the impedance signals are generated based on concentration of inorganic soil carbon and organic soil carbon in the soil sample, wherein the active sensing component is configured to detect a concentration of a carbon complex in the soil sample, and the carbon complex comprises both organic carbon moieties and inorganic carbon moieties.

54. The method of claim 53, further comprising determining, from the impedance signals, a concentration of total soil carbon within the soil sample.

55. The method of claim 53, further comprising transmitting a signal to another computing device to identify the impedance signals to the other computing device, wherein the other computing device determines the concentration of total soil carbon within the soil sample.

56. A composite coating to interact with a carbon complex comprising inorganic carbon and organic carbon, wherein the composite coating comprises:
an organic carbon sensing element functionalized to detect organic carbon moieties;
an inorganic carbon sensing element functionalized to detect inorganic carbon moieties; and
a polymer membrane network.

57. The composite coating of claim 56, wherein the organic carbon sensing element comprises a room temperature ionic liquid (RTIL).

58. The composite coating of claim 57, wherein the RTIL comprises [EMIMTF2N] and the inorganic carbon sensing element comprises calixarene.

59. The composite coating of claim 56, comprising a mixture of the organic carbon sensing element, the inorganic carbon sensing element, and the polymer membrane network

60. The composite coating of claim 56, wherein the composite coating is adapted for screen printing or drop casting onto a surface of a sensor electrode.

61. A system comprising:
a sensor device comprising:
a plurality of electrodes, wherein the plurality of electrodes comprises a working electrode coated in a composite sensing coating, wherein the composite sensing coating comprises an active sensing component functionalized to detect both organic carbon moieties and inorganic carbon moieties and an encapsulant component, and a sealant component, wherein the active sensing component comprises a composite of an organic carbon sensing element and an inorganic carbon sensing element, and the organic carbon sensing element comprises a room temperature ionic liquid (RTIL), wherein the sealant component comprises a polymer membrane network; and
circuitry to generate a signal to identify an impedance or a current based on concentration of total soil carbon in a soil sample when in contact with the plurality of electrodes, wherein the total soil carbon comprises organic soil carbon and inorganic soil carbon; and
an analysis system comprising:
a processor;
analytics logic executable by the processor to determine, from the impedance, a value of the concentration of total soil carbon in the soil sample.

62. The system of claim 61, further comprising a plurality of sensor devices deployed in a plurality of soil samples within an environment.

63. The system of claim 61, wherein the sensor device comprises an in situ soil sensor.

64. The system of claim 61, wherein the analysis system is to collect a series of readings over time and determine changes in the concentration of the total soil carbon from the series of readings.
